(19) United States
(12) Patent Application Publication
(10) Pub. No.: US 2023/0219936 A1
(43) Pub. Date: Jul. 13, 2023

(54) PROCESS FOR THE PREPARATION SALTS OF TRIAZOLE COMPOUNDS

(71) Applicant: Cellix Bio Private Limited, Hyderabad (IN)

(72) Inventor: Mahesh Kandula, East Godavari District, Andhra Pradesh (IN)

(73) Assignee: Cellix Bio Private Limited, Hyderabad (IN)

(21) Appl. No.: 17/925,288

(22) PCT Filed: May 4, 2021
(86) PCT No.: PCT/IB2021/053696

§ 371 Date: Nov. 9, 2022

(30) Foreign Application Priority Data
May 12, 2020 (IN) 202041019951

(51) Int. Cl. C07D 405/14; C07D 403/06

(52) U.S. Cl. CPC C07D 405/14; C07D 403/06; C07B 2200/13

(57) ABSTRACT

The present invention discloses a large scale process for preparation of triazole compounds using polar and non-polar solvents. More particularly relates to s method of preparing dilauryl glyceryl fumarate salt of posaconazole, voriconazole and itraconazole respectively. The method comprises of dissolving triazole compound and dilauryl glyceryl fumarate in a suitable polar solvent at a temperature range of 30-55° C. for salt formation and final salt is isolated using non-polar solvent at a low temperature range of 0-35° C. It further discloses a method of producing fine particulate size of the dilauryl glyceryl fumarate salt of triazole preferably in the size ranging from 0.001 micron to 100 micron. It also discloses a method of preparing a desired pharmaceutical preparation.

### FIELD OF INVENTION

The present invention relates to a process of preparing salts of triazoles compounds, in particular, the invention relates to large scale manufacturing process for preparing dilauryl glyceryl fumarate salt of posaconazole, voriconazole and itraconazole respectively. Further relates to a method of producing these salts for a desired pharmaceutical preparations.

### BACKGROUND OF INVENTION

Invasive fungal infections have been on raise. Despite advancements in antifungal medication treatment, still there are difficulties because of limited administration routes or poor safety profiles. Triazole compounds have been shown to contain some of the most potent antifungal properties. The triazole also known as pyrrodiazole is one of the classes of organic heterocyclic compounds containing a five-membered di unsaturated ring structure composed of three nitrogen atoms and two carbon atoms at non-adjacent positions. The simplest form of the triazole family is triazole itself. Posaconazole, is a triazole antifungal medication sold under the brand names Noxafil and Posanol. Candidiasis is an infection caused by a yeast (a type of fungus) called Candida. The most common is Candida albicans. Candida normally thrives on the skin and inside the body, such as the mouth, throat, gut, and vagina. Candida, cause infections when it grows out of control or if it enters into the bloodstream or internal organs.

Invasive candidiasis occurs when Candida species enter the bloodstream or affect internal organs. Thrush or oropharyngeal candidiasis a condition when candidiasis develops in the mouth or throat. Candidiasis in the vagina is referred to as a yeast infection.

International application WO2018/096405 and WO/2018/096497 discloses various salts and derivatives of triazoles compounds and the methods to manufacturing the same.

Some type of candida are resistant to the antifungals medication or the treatment outcomes were suboptimal due to various factors. Present invention aims at manufacturing salts of triazole which are highly effective with improved potency and lesser toxicity for pharmaceutical preparation and use. The present invention also prepares triazole salts possessing important biological activities.

### SUMMARY OF THE INVENTION

In an embodiment, the present invention discloses a method of preparation of triazole compounds.

In an embodiment the method comprises dissolving triazole compound and dilaurylglyceryl fumarate in a suitable polar solvents for formation of triazole salt at predefined temperature and time. The final salt is isolated using a non-polar cyclic or acyclic hydrocarbon at a low temperature.

In another embodiment the polar solvent for salt formation is selected from polar protic solvents such as methanol, ethanol, isopropanol and water, or polar aprotic solvents like acetonitrile, methyl tertiary butyl ether (MTBE) tetrahydrofuran and chlorinated solvents (like dichloromethane, teracholoethane or chloroform.

In another embodiment, the non-polar solvent for isolation of final product is selected from cyclic and non-cyclic hydrocarbons such as cyclohexane, hexane or heptane.

In an embodiment, present invention particularly discloses the process for preparation of dilauryl glyceryl fumarate salt of posaconazole, voriconazole and itraconazole respectively.

In another embodiment the final salts of dilauryl glyceryl fumarate of posaconazole, dilauryl glyceryl fumarate of voriconazole and dilauryl glyceryl fumarate of itraconazole can be produced in the form of amorphous or crystalline form or a combination.

In another embodiment the present invention discloses a process of producing final salt in particulate state, wherein the process can be chemical or physical.

In yet another embodiment the present invention discloses a triazole salts of a particle size ranging from 0.001 micron to 100 micron.

In another embodiment the present invention discloses method of manipulation such as milling or micronisation for producing desired particle size.

### DETAILED DESCRIPTION OF THE INVENTION

The present invention discloses the process for preparation of dilauryl glyceryl fumarate salt of posaconazole, voriconazole and itraconazole respectively. Salt formation was evaluated using various polar protic solvents (such as methanol, ethanol, isopropanol and water), aprotic solvents like acetonitrile, methyl tertiary butyl ether (MTBE) and chlorinated solvents (like dichloromethane, chloroform etc.) at different temperature (30-55° C.) for different time intervals. The solvent was removed under vacuum to yield the compound as solid. Isolation of final salt was performed using various cyclic and acyclic hydrocarbons (cyclohexane, hexane, heptane etc.) at temperature ranging from 0-35° C.

In an embodiment the triazole salt is processed to a fine material having particle size ranging from 0.001 micron to 100 microns through chemical process or physical manipulation (milling or micronisation).

Posaconazole Dilaurylglyceryl Fumarate

IUPAC name: [4-(4-(((3R,5R)-5-((1H-1,2,4-triazol-1-yl)methyl)-5-(2,4-difluorophenyl)tetrahydrofuran-3-yl)methoxy)phenyl)-1-(4-(1-((2S,S5)-2-hydroxypentan-3-yl)-5-oxo-1,5-dihydro-4H-1,2,4-triazol-4-yl)phenyl) piperazin-1-ium-(E)-4-((1,3-bis(dodecanoyloxy) propan-2-yl)oxy)-4-oxobut-2-enoate]

Reference code: (CLX-SYN-G159-009E).

### Preparation of posaconazole dilauryl glyceryl fumarate

In an embodiment, the posaconazole and dilaurylglyceryl fumarate were suspended in acetonitrile and reaction mixture was heated to 50-55° C. to get a clear solution and temperature was maintained for 3 hr with continuous stirring. Solvent was distilled off at temperature not exceeding 40° C. under vacuum to get a solid. To this hexane was added at room temperature with stirring and then removed under vacuum. Again, hexane was added, and contents were cooled to 0-5° C. and stirred for 60 min, filtered, washed with hexane and dried at 30-45° C. to get white to off-white powder.

During salt formation, various polar solvents (like acetonitrile, isopropanol and water) were evaluated at temperature ranging from 50° C.- to 55° C. Use of acetonitrile has resulted in better yield (87-90%) as compared to other solvents. Hence, acetonitrile is found to be a suitable solvent for dissolution of posaconazole and dilaurylglyceryl fumarate for salt formation.

Use of various non-polar cyclic and acyclic hydrocarbons (cyclohexane, hexane and heptane) was evaluated for isolation of final compound at 0-5° C. Hexane is found to be the best solvent to yield better yield (97%) of the desired compound.

The detailed optimized process for the preparation of Posaconazole dilaurylglyceryl fumarate is described below:

### Example 1

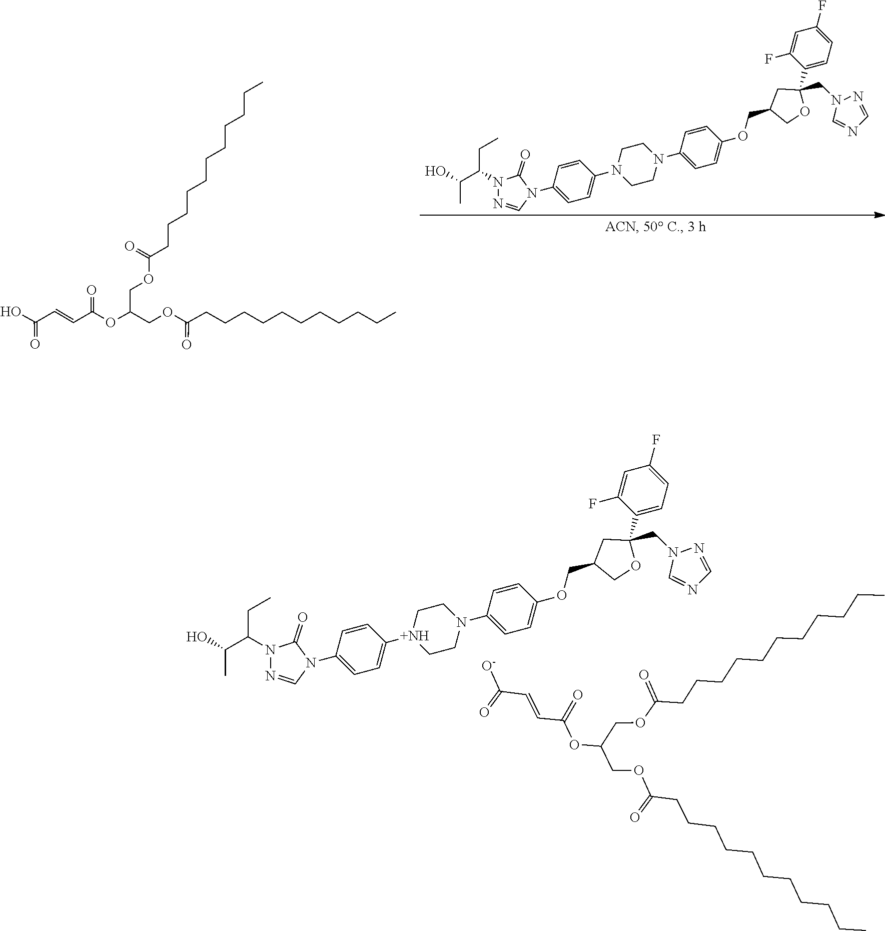 [SMILES: CCCCCCCCCCCC(=O)OCC(COC(=O)CCCCCCCCCCC)OC(=O)/C=C/C(=O)O ; CC[C@@H]([C@H](C)O)n1ncn(-c2ccc(N3CCN(c4ccc(OC[C@@H]5CO[C@@](Cn6cncn6)(c6ccc(F)cc6F)C5)cc4)CC3)cc2)c1=O ; CCC([C@H](C)O)n1ncn(-c2ccc([NH+]3CCN(c4ccc(OC[C@@H]5CO[C@@](Cn6cncn6)(c6ccc(F)cc6F)C5)cc4)CC3)cc2)c1=O ; CCCCCCCCCCCC(=O)OCC(COC(=O)CCCCCCCCCCC)OC(=O)/C=C/C(=O)[O-]]

### Step-I: Preparation of Posaconazole dilauryl glyceryl fumarate

To a solution of Posaconazole (320 g, 0.456 gmole) in acetonitrile (6.4 lt) was added (E)-4-((1,3-bis(didecanoyloxy)propane-2-yl)oxy)-4-oxobut-2-enoic acid (253 gm, 0.456 gmole) at room temperature (25-30° C.). The resulting mixture was heated to 50-55° C. and stirred for 180 minutes at 50-55° C. The contents were filtered, and solvent was removed at temperature not exceeding 40° C. under vacuum to yield a white solid.

### Step-II: Crystallization of Posaconazole dilauryl glyceryl fumarate

The solid was taken in hexane (320 ml) under stirring and solvent was removed under vacuum at 40° C. To the residue, hexane (1.6 lt) was added and contents were cooled to 0-5° C. with continuous stirring and then filtered followed by washing with hexane (320 ml). The solid was dried at 35-40° C. under vacuum to yield the final compound as off white powder (558 g; 97.3% yield).

Chemical Formula: C₆₈H₉₆F₂N₈O₁₂; Molecular Weight: 1255.56;

¹H NMR (400 MHZ, DMSO-d₆ solvent): ¹H NMR chemical shifts are reported on the δ scale in ppm relative to TMS (δ 0.00) as internal reference standard and NMR assignments are shown on the following page:

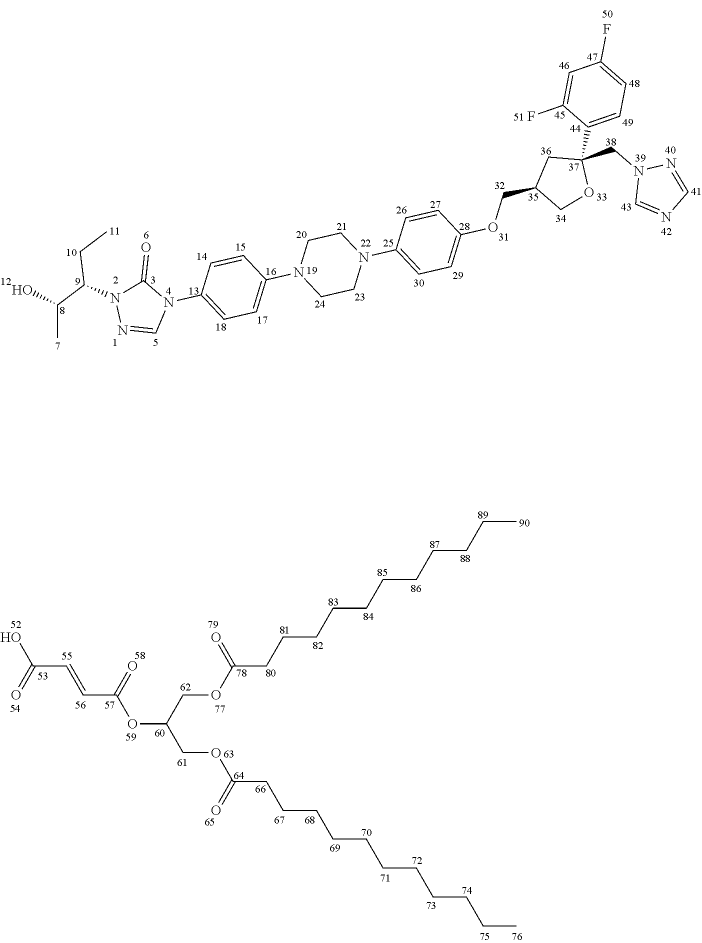 [SMILES: CC[C@@H]([C@H](C)O)n1ncn(-c2ccc(N3CCN(c4ccc(OC[C@@H]5CO[C@@](Cn6cncn6)(c6ccc(F)cc6F)C5)cc4)CC3)cc2)c1=O ; CCCCCCCCCCCC(=O)OCC(COC(=O)CCCCCCCCCCC)OC(=O)/C=C/C(=O)O]

NMR assignments of Posaconazole dilaurylglyceryl fumarate are shown below:

| Position¹ | δ (ppm and Hz) | Position¹ | δ (ppm and Hz) |
|---|---|---|---|
| H5 | 7.77 (s, 1H) | H20, H24, | 3.32-3.14 (m, 8H) |
| | | H21, H23 | |
| H7 | 1.12 (d, 3H) | H26, H30 | 6.93 (d, 2H) |
| H8 | 3.83 (m, 1H) | H27, H29 | 6.79 (d, 2H) |
| H9 | 3.80 (m, 1H) | H32 | 4.02 (t, 1H); |
| | | | 3.66 (t, 1H) |
| H10 | 1.70 (m, 2H) | H34 | 3.69 (m, 1H); |
| | | | 3.63 (m, 1H) |
| H11 | 0.74 (t, 3H) | H35 | 2.53 (m, 1H) |
| H12 | 4.66 (d, 1H) | H36 | 2.13 (m, 1H); |
| | | | 2.39 (m, 1H) |
| H14, H18 | 7.50 (d, 2H) | H38 | 4.57 (m, 2H) |
| H15, H17 | 7.11 (d, 2H) | H41, H43 | 8.34-8.33 (d, 2H) |
| H46 | 7.01 (m, 1H) | H48, H49 | 7.30-7.25 (m, 2H) |
| H52 | 13.27 (s, 1H) | H54, H56 | 6.66 (m, 2H) |
| H61 | 4.22 (m, 2H) | H60 | 5.29 (m, 1H) |
| H66, H80 | 2.28 (t, 4H) | H62 | 4.31 (d, 2H) |
| H76 & H90 | 0.84 (t, 6H) | H67, H81 | 1.46 (m, 4H) |
| H68, H69, H70, | 1.26 (m, 32H) | — | — |
| H71, H72, H73, | | | |
| H74, H75 H82, | | | |
| H83, H84, H85, | | | |
| H86, H87, H88, | | | |
| H89 | | | |

Itraconazole Dilaurylglyceryl Fumarate

IPUAC name: 1-((4-((4-(4-(4-(1-(sec-butyl)-5-oxo-1,5-dihydro-4H-1,2,4-triazol-4-yl)phenyl)piperazin-1-yl)phenoxy)methyl)-2-(2,4-dichlorophenyl)-1,3-dioxolan-2-yl)methyl)-1H-1,2,4-triazol-1-ium-((E)-4-((1,3-bis(dodecanoyloxy)propan-2-yl)oxy)-4-oxobut-2-enoate Ref code: (CLX-SYN-G159-007E)

### Preparation of Itraconazole dilauryl glyceryl fumarate

In an embodiment itraconazole and dilaurylglyceryl fumarate were suspended in dichloromethane and mixture was heated to 35-38° C. to get a clear solution. Reaction temperature was maintained for 3 hr with continuous stirring. The solvent was distilled off to get a solid. To this hexane was added with stirring at 25-30° C. and removed under vacuum. This solid was taken in hexane and contents were stirred at 25-30° C. and cooled to 0-5° C., filtered and washed with hexane. The material was dried at 45-50° C. to get off-white to light yellow powder.

During salt formation, various polar solvents (like acetonitrile, tetrahydrofuran and chlorinated solvent (such as dichloromethane) were evaluated at temperature ranging from 35° C.- to 55° C. Use of acetonitrile did not result in a clear solution. Though a clear solution was obtained when salt formation was performed in tetrahydrofuran/dichloromethane. Dichloromethane has resulted in good yield (97%) as compared to THF (95%). Hence, dichloromethane is found to be a suitable solvent for dissolution of Iitraconazole and dilaurylglyceryl fumarate during for salt formation.

Effect of reaction duration during salt formation was evaluated by maintaining the reaction temperature for different time intervals (30 min to 5 hr). Maintaining the reaction temperature for 3 hr has resulted in better yield.

Use of various non-polar cyclic and acyclic hydrocarbons (cyclohexane, hexane and heptane) was evaluated for isolation of final compound. Hexane is found to be the best solvent to yield better yield (95%) of the desired compound.

Effect of temperature on isolation of compound using hexane as a solvent was studied at 0-15° C. and 25-30° C. Isolation of final compound at lower temperature (0-5° C.) has resulted in good yield (96%) as compared to higher temperature.

The detailed optimized process for the preparation of Itraconazole dilaurylglyceryl fumarate is described on the following page:

### Example 2

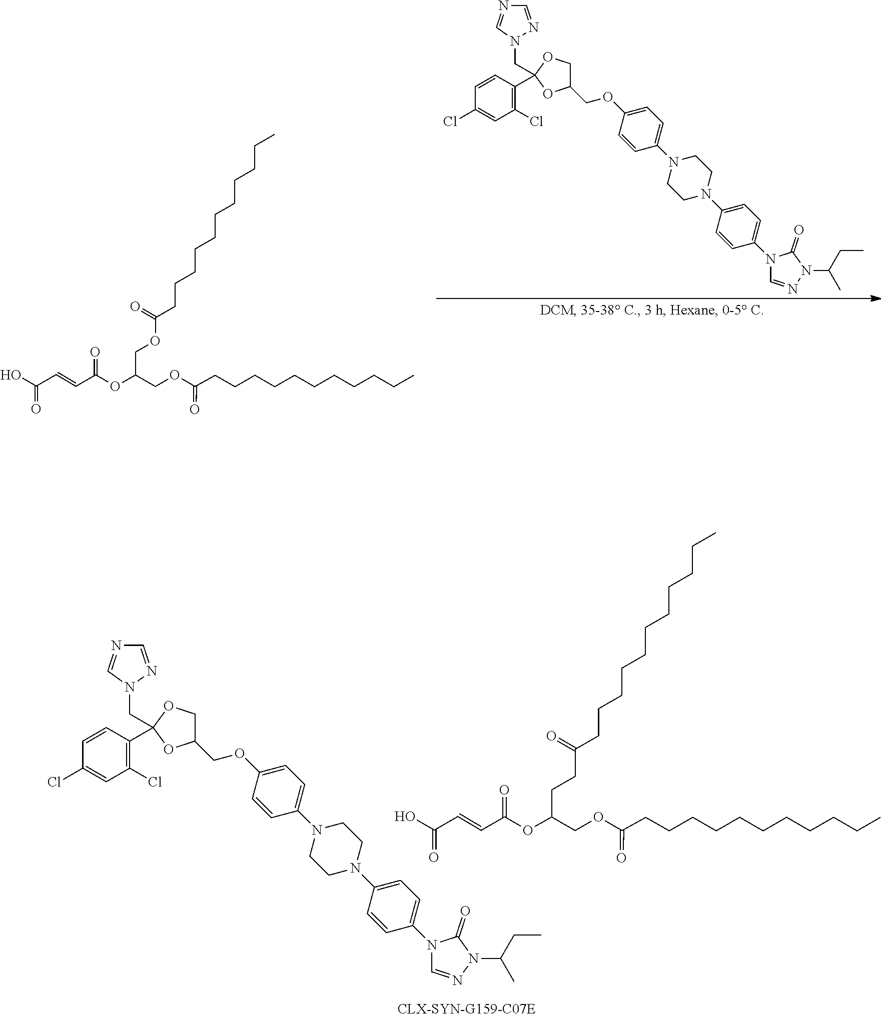 [SMILES: CCCCCCCCCCCC(=O)OCC(COC(=O)CCCCCCCCCCC)OC(=O)/C=C/C(=O)O ; CCC(C)n1ncn(-c2ccc(N3CCN(c4ccc(OCC5COC(Cn6cncn6)(c6ccc(Cl)cc6Cl)O5)cc4)CC3)cc2)c1=O ; CCC(C)n1ncn(-c2ccc(N3CCN(c4ccc(OCC5COC(Cn6cncn6)(c6ccc(Cl)cc6Cl)O5)cc4)CC3)cc2)c1=O ; CCCCCCCCCCCC(=O)CCC(COC(=O)CCCCCCCCCCC)OC(=O)/C=C/C(=O)O]

### Step-I: Preparation of Itraconazole dilauryl glyceryl fumarate

Itraconazole (270 gm, 0.382 gmole) was added to solution of (E)-4-((1,3-bis(didecanoyloxy)propanes-2-yl)oxy)-4-oxobut-2-enoic acid (212.22 g, 0.382 gmole) in dichloromethane (2.7 lt) at 25-30° C. The resulting solution was heated to reflux (35-38° C.) with continuous stirring for 120 min. This was given carbon treatment for 60 min and filtered through hyflo bed followed by washing with dichloromethane (1.35 lt). The solvent was removed under vacuum at temperature not exceeding 40° C.

### Step-II: Crystallisation of Itraconazole dilaurylglyceryl fumarate

The solid was taken in hexane (1.08 lt) under stirring and solvent was removed under vacuum at 40° C. To the residue, hexane (3.78 lt) was added and contents were cooled to 0-5° C. with continuous stirring and then filtered followed by washing with hexane (1.08 lt). The solid was dried at 45-50° C. under vacuum to yield the final compound as light yellow solid (476 gm, yield 98.7%).

Chemical Formula: C₆₆H₉₂Cl₂N₈O₁₂; Molecular Weight: 1260.41 ¹H NMR (400 MHZ, DMSO-d₆ solvent): ¹H NMR chemical shifts are reported on the δ scale in ppm relative to TMS (δ 0.00) as internal reference standard and NMR assignments are shown on the following page:

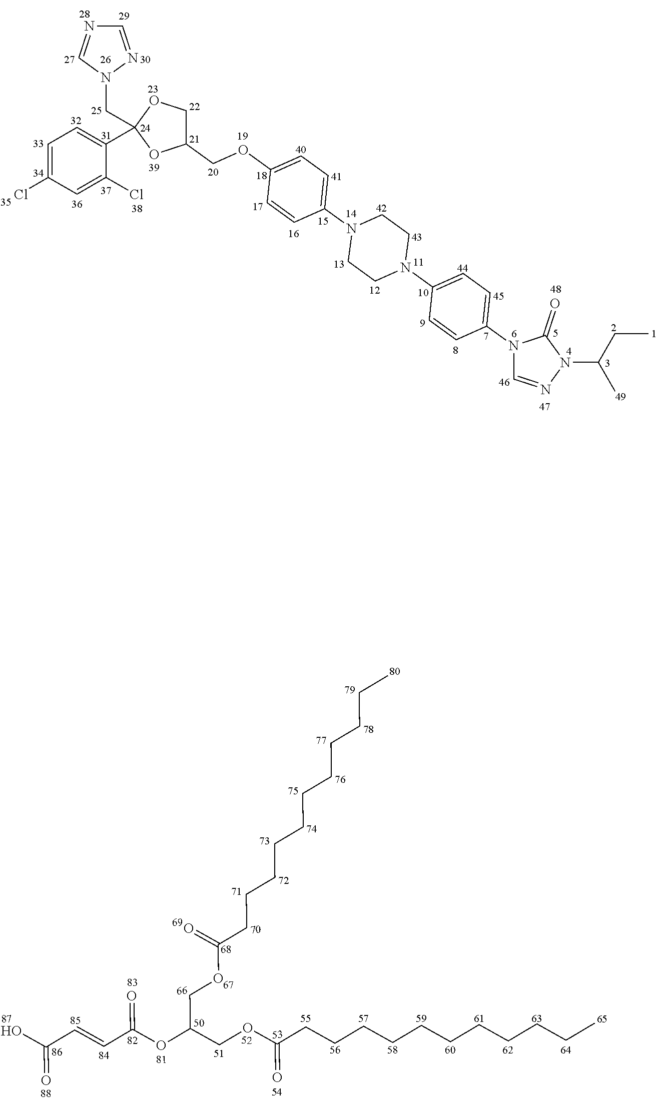 [SMILES: CCC(C)n1ncn(-c2ccc(N3CCN(c4ccc(OCC5COC(Cn6cncn6)(c6ccc(Cl)cc6Cl)O5)cc4)CC3)cc2)c1=O ; CCCCCCCCCCCC(=O)OCC(COC(=O)CCCCCCCCCCC)OC(=O)/C=C/C(=O)O]

NMR assignments of Itraconazole dilaurylglyceryl fumarate are shown below:

| Position¹ | δ (ppm and Hz) | Position¹ | δ (ppm and Hz) |
|---|---|---|---|
| H1 | 0.79 (t)-3H | H32 | 7.67 (d)-1H |
| H2 | 1.22 (m)-2H | H33 | 7.41 (dd)-1H |
| H3 | 3.92 (m)-1H | H36 | 7.67 (d)-1H |
| H8 & H45 | 7.10 (d)-2H | H46 | 7.87 (s)-1H |
| H9 & H44 | 6.97 (d)-2H | H49 | 1.26 (m)-3H |
| H12 & H43 | 3.19 (m)-4H | H50 | 4.82 (m)-1H |
| H13 & H42 | 3.33 (m)-4H | H51 & H66 | 4.35 (m)-2H, |
| | | | 4.31 (m)-2H |
| H16 & H41 | 6.85 (d)-2H | H55 & H70 | 2.28 (m)-4H |
| H17 & H40 | 6.67 (q)-2H | H56 & H71 | 1.47 (m)-4H |
| H20 | 3.92(m)-1H; | H57, H58, H59, | 1.22 (m)-32H |
| | 4.92(m)-1H | H60, H61, H62, | |
| | | H63, H64, H72, | |
| | | H73, H74, H75, | |
| | | H76, H77, H78, | |
| | | H79 | |
| H21 | 4.81 (m)-1H | H65 & H80 | 081 (m)-6H |
| H22 | 3.75 (m)-2H | H84 | 7.41 (d)-1H |
| H25 | 4.24 (m)-2H | H85 | 7.52 (d)-1H |
| H27 | 8.44 (s)-1H | H87 | 13.21 (s)-1H |
| H29 | 8.41 (s)-1H | — | — |

Voriaconazole Dilaurylglyceryl Fumarate

IUPAC NAME: [4-((2S,3R)-3-(2,4-difluorophenyl)-3-hydroxy-4-(1H-1,2,4-triazol-1-yl)butan-2-yl)-5-fluoropyrimidin-1-ium-(E)-4-((1,3-bis(dodecanoyloxy)propan-2-yl)oxy)-4-oxobut-2-enoate] (CLX-SYN-G159-C10E):

### Preparation of Voriconazole dilauryl glyceryl fumarate

In another embodiment the voriconazole and dilaurylglyceryl fumarate were suspended in an acetonitrile and contents were heated to 50-55° C. to get a clear solution and stirring was continued for 3 hr. Solvent was distilled off to get a solid. To this hexane was added with stirring at 25-30° C. and removed under vacuum. This solid was taken in hexane and contents were stirred at 25-30° C. and cooled to 0-5° C., filtered and washed with hexane. The material was dried at 35-40° C. to get white to off-white solid.

During preparation of salt formation, various polar protic solvents (like Methanol, Ethanol, Isopropanol and Water) and aprotic solvents (acetonitrile, chlorinated solvent (dichloromethane, chloroform) were used at temperature ranging from 40° C.- to 55° C. Use of acetonitrile has resulted in good yield.

Use of various polar (Acetonitrile, Methanol, Isopropanol, Methyl tertiary butyl ether (MTBE) and Water) and non-polar solvents [cyclic and acyclic hydrocarbons (cyclohexane, hexane and heptane)] was studied. Hexane is found to be the best solvent to yield better yield (95%) of the desired compound.

Effect of temperature on isolation of compound using hexane as a solvent was studied at 0-15° C. and isolation at lower temperature (0-5° C.) has resulted in good yield (96%) as compared to higher temperature.

The detailed optimized process for the preparation of Voriconazole dilaurylglyceryl fumarate is described below:

### Example 3

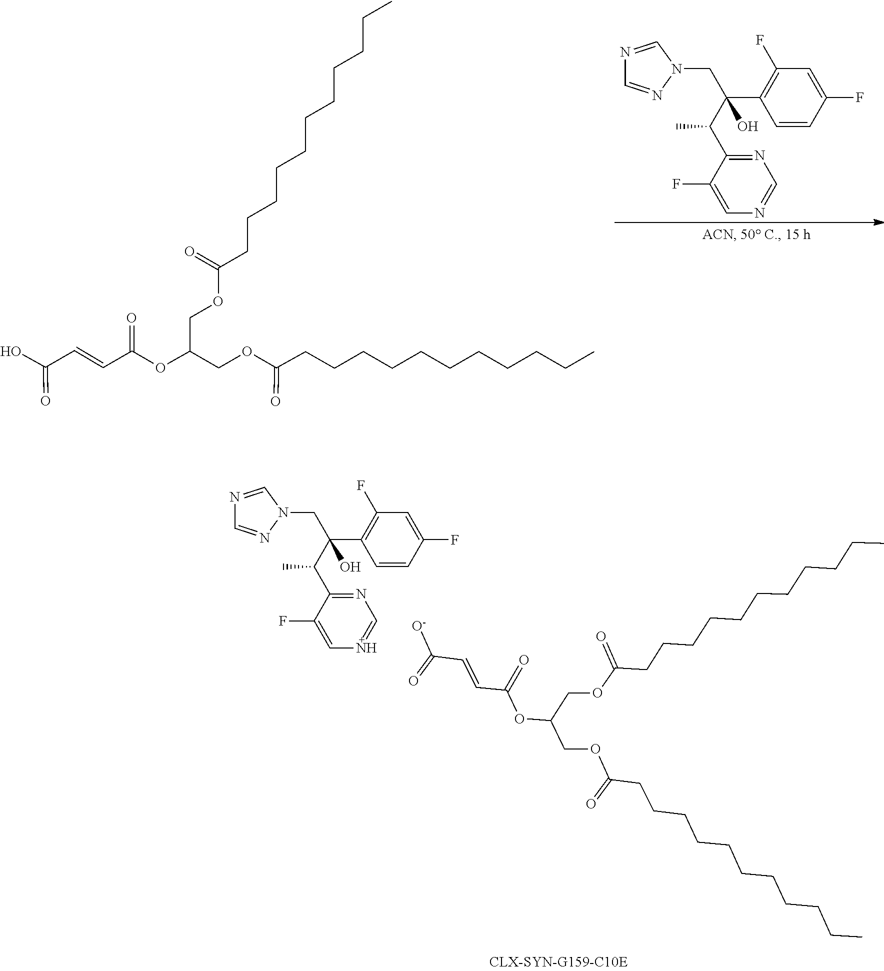 [SMILES: CCCCCCCCCCCC(=O)OCC(COC(=O)CCCCCCCCCCC)OC(=O)/C=C/C(=O)O ; C[C@@H](c1ncncc1F)[C@](O)(Cn1cncn1)c1ccc(F)cc1F ; C[C@@H](c1ncncc1F)[C@](O)(Cn1cncn1)c1ccc(F)cc1F ; CCCCCCCCCCCC(=O)OCC(COC(=O)CCCCCCCCCCC)OC(=O)/C=C/C(=O)[O-]]

### Step-I: Preparation of Voriconazole dilauryl glyceryl fumarate

Voriconazole (230 gm, 0.658 gmole) was added to a solution of (E)-4-((1,3-bis(didecanoyloxy)propane-2-yl)oxy)-4-oxo but-2-enoic acid (365.5 gm, 0.658 gmole) in acetonitrile (6.9 lt) at room temperature (25-35° C.). The resulting mixture was heated to 50-55° C. and stirred for 180 minutes at 50-55° C. The mixture was filtered, and solvent was removed under vacuum at temperature not exceeding 40° C.

### Step-II: Crystallization of Voriconazole dilauryl glyceryl fumarate

The solid was taken in hexane (460 ml) under stirring at 25-30° C. and solvent was removed under vacuum at 40° C. To the residue, hexane (1.1510 was added under stirring and contents were cooled to 0-5° C., stirred for 60 min and then filtered. The solid was washed with hexane (230 ml) and material was dried at 35-40° C. under vacuum to yield the final compound as off white powder (588 g; 98.8% yield).

Chemical Formula: C₄₇H₆₈F₃N₅O₉; Molecular Weight: 904.09

¹H NMR (400 MHZ, DMSO-d₆ solvent): 1H NMR chemical shifts are reported on the δ scale in ppm relative to TMS (δ 0.00) as internal reference standard and NMR assignments are shown as below.

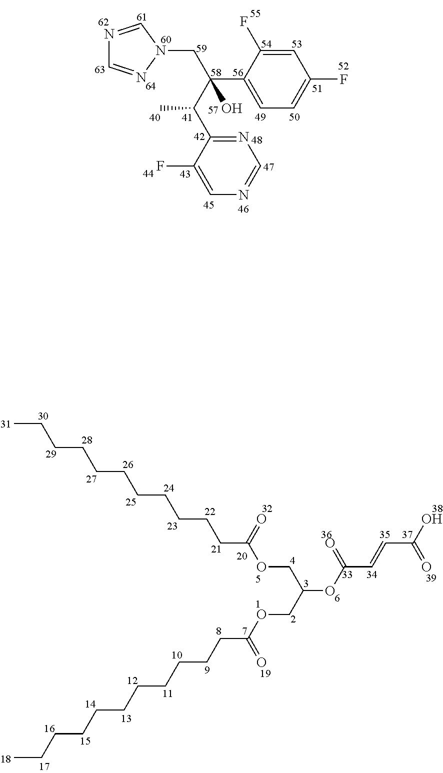 [SMILES: C[C@@H](c1ncncc1F)[C@](O)(Cn1cncn1)c1ccc(F)cc1F ; CCCCCCCCCCCC(=O)OCC(COC(=O)CCCCCCCCCCC)OC(=O)/C=C/C(=O)O]

NMR assignments of Voriconazole dilaurylglyceryl fumarate are shown below:

| Position¹ | δ (ppm and Hz) | Position | δ (ppm and Hz) |
|---|---|---|---|
| H2 | 4.22 (d, 2H) | H10, H11, H12, H13, | 1.26 (m, 32H) |
| | | H14, H15, H16, H17, | |
| | | H23, H24, H25, H26, | |
| | | H27, H28, H29, H30 | |
| H3 | 5.29 (m, 1H) | H18 & H31 | 0.84 (t, 6H) |
| H4 | 4.31 (m, 2H) | H34, H35 | 6.66 (m, 2H) |
| H8, H21 | 2.28 (t, 4H) | H38 | 13.27 (s, 1H) |
| H9, H22 | 1.46 (m, 4H) | H41 | 3.95 (q, 1H) |
| H40 | 1.13 (d, 3H) | H47 | 9.06 (d, 2H) |
| H45 | 8.87 (d, 1H) | H50 | 7.17 (m, 1H) |
| H49 | 7.27 (m, 1H) | H57 | 5.99 (s, 1H) |
| H53 | 6.92 (m, 1H) | H61 | 8.24 (s, 1H) |
| H59 | 4.37 (d, 1H); | H63 | 7.62 (s, 1H) |
| | 4.83 (d, 1H) | | |

We claim:

1. A process for preparation of triazole salts comprises of:
a. the salt formation; by dissolving a triazole compound and a dilaurylglyceryl fumarate in a suitable polar solvents for formation of a triazole salt at a pre-determined temperate and time; and
b. isolation of the triazole salt using a non-polar cyclic or acyclic hydrocarbon solvent; wherein the polar solvent for salt formation is selected from polar protic solvents or polar aprotic solvents; further the non-polar solvent for isolation of the triazole salt is selected from cyclic and acyclic hydrocarbons.

2. The process of claim 1, wherein the triazole compounds are posaconazole, voriconazole and itraconazole.

3. The process of claim 1, wherein the polar protic solvents and polar aprotic solvents for salt formation comprises methanol, ethanol, isopropanol, water, acetonitrile, methyl tertiary butyl ether (MTBE), tetrahydrofuran, chlorinated solvent, dichloromethane, teracholoethane or chloroform preferably acetonitrile and dichloromethane.

4. The process of claim 1, wherein, the non-polar cyclic or acyclic hydrocarbon solvent for isolation comprises cyclohexane, hexane or heptane preferably hexane.

5. The process of claim 1, wherein, the triazole salt are isolated in the form of amorphous salt, crystalline salt or a combination.

6. The process of claim 1, wherein the triazole salts are selected from a group consisting of dilauryl glyceryl fumarate of posaconazole, dilauryl glyceryl fumarate of voriconazole and dilauryl glyceryl fumarate of itraconazole.

7. The process according to claim 1, wherein the preparation of dilauryl glyceryl fumarate of posaconazole comprises of:
a. dissolving posaconazole and dilaurylglyceryl fumarate in the acetonitrile and heating the reaction mixture to a temperature about 50-55° C. for 180 minutes with continuous stirring to obtain a clear solution;
b. acetonitrile is distilled off from the reaction mixture at temperature not exceeding 40° C. to obtain a solid having the dilauryl glyceryl fumarate of posaconazole; and
c. dilauryl glyceryl fumarate of posaconazole is isolated from the solid of step b by washing and filtering the solid in hexane to yield above 97.3% dilauryl glyceryl fumarate of posaconazole a solid.

8. The process as claimed in claim 1, wherein the preparation of dilauryl glyceryl fumarate of voriconazole comprises of:
a. dissolving voriconazole and dilaurylglyceryl fumarate in the acetonitrile and heating the reaction mixture to a temperature about 50-55° C. for 180 minutes with continuous stirring to obtain a clear solutions;
b. acetonitrile is distilled off from the reaction mixture at temperature not exceeding 40° C. to obtain a solid mixture having the dilauryl glyceryl fumarate of voriconazole; and
c. dilauryl glyceryl fumarate of voriconazole is isolated from the solid mixture by washing, filtering drying the solid mixture in hexane to yield above 98.8% dilauryl glyceryl fumarate of voriconazole as a solid.

9. The process as claimed in claim 1, wherein the preparation of dilauryl glyceryl fumarate of itraconazole comprises of:
a. dissolving itraconazole and dilaurylglyceryl fumarate in the dichloromethane and heating the reaction mixture to a temperature about 35-38° C. for 120 minutes with continuous stirring, this mixture is subjected to carbon treatment for 60 minute and the mixture is filtered through a hyflo bed and washed with dichloromethane;
b. dichloromethane is distilled off from the reaction mixture at temperature not exceeding 40° C. to obtain a solid mixture having the dilauryl glyceryl fumarate of itraconazole; and
c. dilauryl glyceryl fumarate of itraconazole is isolated from the solid mixture by washing, filtering drying the solid mixture in hexane to yield above 98.7% dilauryl glyceryl fumarate of itraconazole as a solid.

10. The triazole salts prepared by the process according to claim 7, are processed using a chemical process or physical manipulation to obtain a particle size ranging from 0.001 micron to 100 micron through.

11. The triazole salts prepared by the process according to claim 8, are processed using a chemical process or physical manipulation to obtain a particle size ranging from 0.001 micron to 100 micron through.

12. The triazole salts prepared by the process according to claim 9, are processed using a chemical process or physical manipulation to obtain a particle size ranging from 0.001 micron to 100 micron through.

13. The process according to claim 6, wherein the preparation of dilauryl glyceryl fumarate of posaconazole comprises of:
a. dissolving posaconazole and dilaurylglyceryl fumarate in the acetonitrile and heating the reaction mixture to a temperature about 50-55° C. for 180 minutes with continuous stirring to obtain a clear solution;
b. acetonitrile is distilled off from the reaction mixture at temperature not exceeding 40° C. to obtain a solid having the dilauryl glyceryl fumarate of posaconazole; and
c. dilauryl glyceryl fumarate of posaconazole is isolated from the solid of step b by washing and filtering the solid in hexane to yield above 97.3% dilauryl glyceryl fumarate of posaconazole.

14. The process as claimed in claim 6, wherein the preparation of dilauryl glyceryl fumarate of voriconazole comprises of:
a. dissolving voriconazole and dilaurylglyceryl fumarate in the acetonitrile and heating the reaction mixture to a temperature about 50-55° C. for 180 minutes with continuous stirring to obtain a clear solution;
b. acetonitrile is distilled off from the reaction mixture at temperature not exceeding 40° C. to obtain a solid mixture having the dilauryl glyceryl fumarate of voriconazole; and
c. dilauryl glyceryl fumarate of voriconazole is isolated from the solid mixture by washing, filtering drying the solid mixture in hexane to yield above 98.8% dilauryl glyceryl fumarate of voriconazole as a solid.

15. The process as claimed in claim 6, wherein the preparation of dilauryl glyceryl fumarate of itraconazole comprises of:
a. dissolving itraconazole and dilaurylglyceryl fumarate in the dichloromethane and heating the reaction mixture to a temperature about 35-38° C. for 120 minutes with continuous stirring, this mixture is subjected to carbon treatment for 60 minute and the mixture is filtered through a hyflo bed and washed with dichloromethane;
b. dichloromethane is distilled off from the reaction mixture at temperature not exceeding 40° C. to obtain a solid mixture having the dilauryl glyceryl fumarate of itraconazole; and
c. dilauryl glyceryl fumarate of itraconazole is isolated from the solid mixture by washing, filtering drying the solid mixture in hexane to yield above 98.7% dilauryl glyceryl fumarate of itraconazole as a solid.

16. The triazole salts prepared by the process according to claim 13, are processed using a chemical process or physical manipulation to obtain a particle size ranging from 0.001 micron to 100 micron through.

17. The triazole salts prepared by the process according to claim 14, are processed using a chemical process or physical manipulation to obtain a particle size ranging from 0.001 micron to 100 micron through.

18. The triazole salts prepared by the process according to claim 15, are processed using a chemical process or physical manipulation to obtain a particle size ranging from 0.001 micron to 100 micron through.
